(19) United States
(12) Patent Application Publication
(10) Pub. No.: US 2019/0255114 A1
(43) Pub. Date: Aug. 22, 2019

(54) STORAGE OF PLATELETS

(71) Applicant: University of Kentucky Research Foundation, Lexington, KY (US)

(72) Inventors: Zhenyu Li, Lexington, KY (US); Binggang Xiang, Lexington, KY (US); Guoying Zhang, Lexington, KY (US)

(73) Assignee: University of Kentucky Research Foundation, Lexington, KY (US)

(21) Appl. No.: 16/273,340

(22) Filed: Feb. 12, 2019

Related U.S. Application Data
(60) Provisional application No. 62/710,468, filed on Feb. 16, 2018.

(51) Int. Cl. A61K 35/19; A61P 7/04; A01N 1/02; A01K 67/027

(52) U.S. Cl. CPC A61K 35/19; A61P 7/04; A01N 1/0226; A01K 2227/105; A01K 2217/206; A01K 2267/025; A01K 2267/0393; A01K 67/0275

(57) ABSTRACT

The present disclosure describes platelet compositions for storage and methods of storing platelet compositions in the cold and room temperature. The platelet compositions including platelets and one or more Ca⁺⁺ chelators can be stored in the cold for longer than three days. The platelet compositions can be used to treat subjects with a platelet disease or disorder.

### CROSS REFERENCE TO RELATED PATENT APPLICATIONS

This application claims the benefit of U.S. Provisional Application 62/710,468, filed on Feb. 16, 2018, which is hereby incorporated by reference in its entirety.

### TECHNICAL FIELD

The present disclosure relates to storage of platelets, particularly cold storage of platelets.

### BACKGROUND

Platelets are thrombocytes which are a component of blood. Mammalian platelets have no cell nucleus. They are composed of fragments of cytoplasm derived from the megakaryocytes of the bone marrow. When activated, platelets aggregate to stop the flow of blood from damaged blood vessels. Thus, their main function is to aid in the blood clotting process.

Platelets play essential roles in hemostasis. Platelet transfusion is a life-saving treatment to prevent or treat bleeding in thrombocytopenic patients or patients with platelet dysfunction. Platelet transfusion is also used in trauma patients to prevent potential bleeding. There are more than two million platelet products administered annually in the United States, and the demand for platelet transfusions is rising each year. The annual global market for platelet products is $20 billion.

Currently, most platelet products for transfusion in the United States are stored in di-(2-ethylhexyl) phthalate (DEHP) plasticized polyvinyl chloride bags at 20° C. to 24° C. with gentle agitation. However, room temperature (RT) storage has a high risk of bacterial contamination. It is estimated that 1 in 2000 to 1 in 1000 platelet products are contaminated by bacteria, which could cause sepsis and death in recipients after transfusion. The risk of bacterial infection of the platelet products is 50 times more than that of refrigerated red blood cells. Overall, bacterial contamination is the second most common cause of death from transfusion in the United States.

Another major problem from RT storage is the loss of platelet hemostatic function due to platelet storage lesions (PSLs) by increased metabolism. Platelets stored at RT undergo a series of changes in morphology and function, including loss of the discoidal shape, release of granule contents, phosphatidylserino (PS) exposure, and modifications of glycoprotein patterns on the surface.

Because of these two major problems, the current shelf-life of platelet products is limited to 5 days and a testing for bacterial contamination is required. Some hospitals even have stricter criteria and do not use platelets stored over three days. The shelf-life is markedly shorter compared to red blood cells, which are stored for 45 days in the refrigerator. The short storage time and bacterial contamination result in discarding one quarter of the platelet products, which amount to over $100 million in losses annually in the United States and results in a continued shortage of platelet products in blood transfusion services globally.

Accordingly, there is a need to develop an improved method of storing platelets.

### SUMMARY

The present disclosure provides a novel method for storing platelets in the cold. The method includes adding one or more Ca⁺⁺ chelators (calcium chelators) to a platelet composition to extend the shelf-life of the composition. The present disclosure describes platelet compositions that can be stored in the cold for longer than three days. The platelet compositions include platelets and one or more Ca⁺⁺ chelators. In embodiments, the platelet composition includes plasma rich platelet (PRP) and one or more Ca⁺⁺ chelators.

The present disclosure also provides methods of using the platelet compositions described herein to treat platelet diseases or disorders. In embodiments, the platelet compositions described herein can stop bleeding faster than platelet composition stored at RT.

### BRIEF DESCRIPTION OF THE DRAWINGS

FIGS. 1A, 1B, and 1C show cold induces platelet aggregation. (A) and (B) show cold-induced platelet aggregation and reduction in single platelet counts in washed platelets. (A) Washed platelets from mice were stored at RT or 4° C. for 24 hours. (B) Washed platelets from C57BL/6 mice were stored at RT or 4° C. for various time and single platelet counts were monitored. (C) O-sialoglycoprotein endopeptidase inhibited cold-induced platelet aggregation.

FIGS. 2A, 2B, and 2C show the effect of cold storage on platelets. (A) and (B) show cold storage elicited integrin activation. (C) shows cold storage elicited Src phosphorylation.

FIGS. 3A and 3B show cold storage elicited platelet secretion. (A) and (B) Serotonin (A) and PF4 (B) secreted into supernatant were measured.

FIG. 4 shows cold-induced aggregation was abolished in the integrin β3 deficient platelets. Washed platelets from β^{-/-} mice were stored at RT or 4° C. for various time.

FIGS. 5A and 5B show cold-induced platelet activation was inhibited by RGDS peptide. Washed platelets from C57BL/6 mice were added with 1 mM RGDS and stored 4° C. for 24 hours.

FIGS. 6A and 6B show a novel assay for detecting life span of transfused platelets. C57BL/6J mice were injected retro-orbitally with washed GFP platelets (2.5×10⁸ per mouse). 60 μl blood was collected from mice and PRP was analyzed by flow cytometry. Quantitative results were expressed as percentage of survived transfused platelets (GFP positive cells at 5 min after transfusion was set to 100%; mean±SD; n=5).

FIG. 7 shows RGDS peptide inhibited rapid clearance of cold-stored platelets. C57BL/6J mice were injected retro-orbitally with fresh washed GFP platelets or GFP platelets stored at 4° C. for 24 hours (2.5×10⁸ per mouse) with or without 1 mM RGDS. Blood was collected from mice and platelets were analyzed by flow cytometry. Quantitative results were expressed as percentage of survived transfused platelets (GFP positive cells of transfused fresh platelets at 5 min after transfusion was set to 100%, n=4).

FIG. 8 shows EGTA prevented reduction in platelet counts during cold storage. Washed platelets from C57BL/6 mice, in the presence or absence of EGTA, were stored at RT or 4° C. for various time.

FIG. 9 shows EGTA inhibited rapid clearance of cold-stored platelets. C57BL/6J mice were injected retro-orbitally with fresh washed GFP platelets or GFP platelets stored at 4° C. for 24 hours (2.5×10⁸ per mouse) with or without 50 uM EGTA. Blood was collected from mice, and platelets were analyzed by flow cytometry. Quantitative results were expressed as percentage of survived transfused platelets (GFP positive cells of transfused fresh platelets at 5 min after transfusion was set to 100%, n=4).

FIGS. 10A and 10B show the effect of EGTA treatment on platelets in PRP. (A) EGTA inhibited clearance of cold stored platelets in PRP. (B) EGTA prevented reduction in platelet counts by cold storage in PRP.

FIGS. 11A and 11B show cold-stored platelets protected against bleeding in GPIbα deficient mice.

FIGS. 12A, 12B, 12C, and 12D show that the effect of EGTA on platelets is reversible. (A) shows EGTA reversibly inhibited platelet aggregation. (B) shows EGTA reversibly inhibited platelet spreading on fibrinogen. (C) and (D) show EGTA reversibly inhibited integrin ligand binding function.

FIGS. 13A and 13B show that the platelets lost their function after RT storage for two days.

FIGS. 14A, 14B, 14C, and 14D show cold storage induces activation and aggregation of human platelets. (A) EGTA and RGDS inhibited the decrease in platelet counts decreased with storage at 4° C. (B) Fibrinogen binding to platelets increased after storage at 4° C. (C) Storage at 4° C. induces platelet secretion of serotonin, which is inhibited by EGTA or RGDS. (D) Storage at 4° C. induces Src phosphorylation.

FIGS. 15A and 15B show that EGTA treated human platelets stored in the cold are better able to maintain their hemostatic function than platelets stored at RT as shown by their response to ADP. (A) Platelets stored at RT lost their function and did not respond to ADP after three days. In contrast, EGTA treated platelets stored at 4° C. were able to preserve their function and respond to ADP even after 9 days. (B) In the presence of ADP, platelets stored at RT lost their ability to aggregate as compared to EGTA treated platelets stored at 4° C.

FIGS. 16A and 16B show that EGTA treated human platelets stored in the cold are better able to maintain their hemostatic function than platelets stored at RT as shown by their response to the PAR1 peptide. (A) EGTA treated platelets stored at 4° C. maintained their ability to respond the PAR1 peptide even after 11 days. In contrast, RT stored platelets lost their ability to respond to the PAR1 peptide after 3 days. (B) In the presence of the PAR1 peptide, platelets stored at RT started losing their ability to aggregate sooner as compared to EGTA treated platelets stored at 4° C.

### DETAILED DESCRIPTION

In the past six decades, researchers have spent tremendous effort in developing new techniques to improve platelet storage. Storing platelets in the cold is believed to minimize bacterial contamination and reduce platelet metabolism, thereby prolonging the storage time and reducing waste. Indeed, cold storage has been shown to decrease lactate accumulation and better preserve platelet aggregation response in vitro compared with RT storage. Thus, platelets were stored in the cold before late 1960s. However, because it was reported in the late 1960s that chilled platelets are cleared rapidly from circulation after transfusion, platelets have not been stored in the cold thereafter.

The mechanism by which chilled platelets are cleared rapidly has not been fully understood. Despite rapid clearance in vivo, the FDA and the AABB approved the use of 3-day cold-stored platelets without agitation and bacterial testing for patients with actively bleeding trauma in 2015 because of the advantages of cold-stored platelets over RT stored platelets. Unfortunately, this approved cold storage method has up to 80.9% disposal rate because of the short 3-day storage time and formation of clots in cold-stored platelets. To date, no method has been successfully developed to allow platelets storage in cold over three days.

Storage of platelets in the cold is preferred over RT storage because it minimizes bacterial contamination and reduces platelet metabolism. However, platelets are not stored in the cold because cold-stored platelets are rapidly cleared from circulation after transfusion. Accordingly, the ideal method for platelet storage must meet the following conditions: (i) platelets remain active; and (ii) patient safety. Moreover, the platelet count must remain constant during storage.

Understanding the mechanism that leads to rapid clearance by cold storage would be helpful for developing a method for storing platelet in the cold. The present disclosure provides insights into the mechanism of rapid clearance after cold storage.

Glycoprotein (GP) IIb/IIIa (integrin alpha-IIb/beta-3) is a platelet specific integrin complex. This complex is a receptor for fibrinogen and von Willebrand factor and is involved in platelet activation. When platelets are activated, the granules in the platelets secrete clotting mediators such as ADP and thromboxane A2, which bind their receptors on the surface of platelets. The binding of the receptors further leads to integrin GPIIb/IIIa activation. Integrin GPIIb/IIIa is transformed from a low-affinity state to a high-affinity state for its ligands including fibrinogen. The binding of fibrinogen to GPIIb/IIIa complex bridges platelets and forming a clot.

Platelets contain dense granules, and upon activation, they secrete small molecules such as serotonin, ADP, and polyphosphates. Platelets also contain alpha granules which secrete various proteins including hemostatic factors (fibrinogen, Factor V), angiogenic factors (VEGF, angiogenin), anti-angiogenic factors (PF4, angiostatin), growth factors (PDGF, bFGF), proteases (MMP2, MMP9), necrotic factors (TNFα, TNFβ), and other cytokines. Platelet secretion upon activation is a normal response to vascular damage.

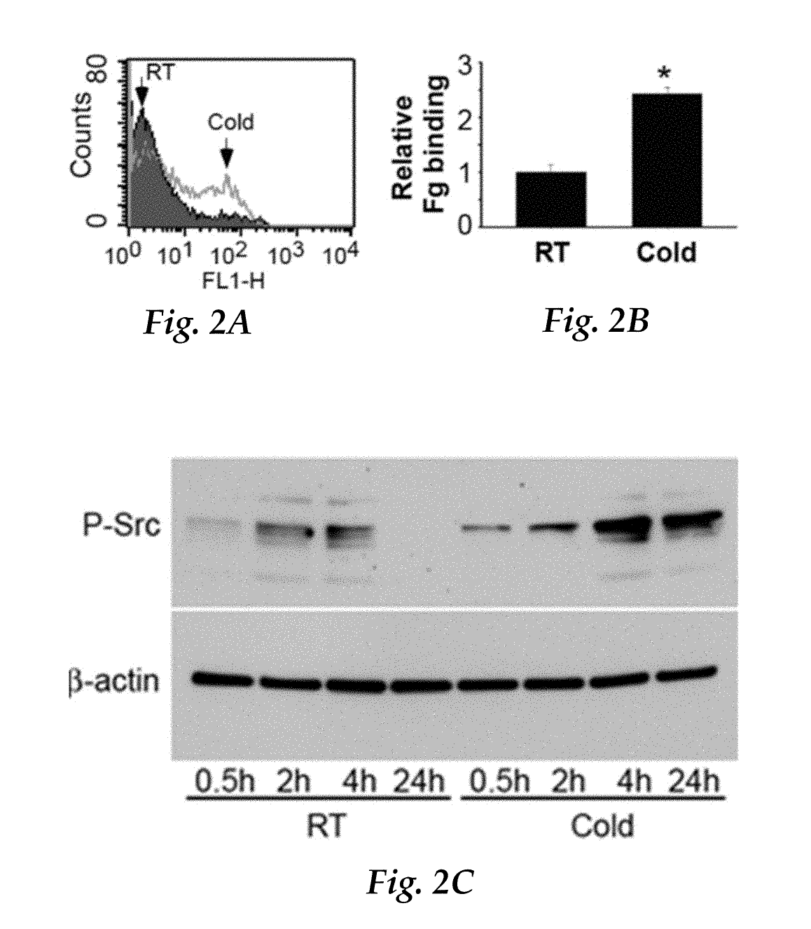

The present disclosure shows that cold storage of platelets induces platelet aggregation which is the consequence of integrin activation. As shown in FIGS. 2A and 2B, there is an increase in fibrinogen binding to platelets under cold storage as compared to RT storage.

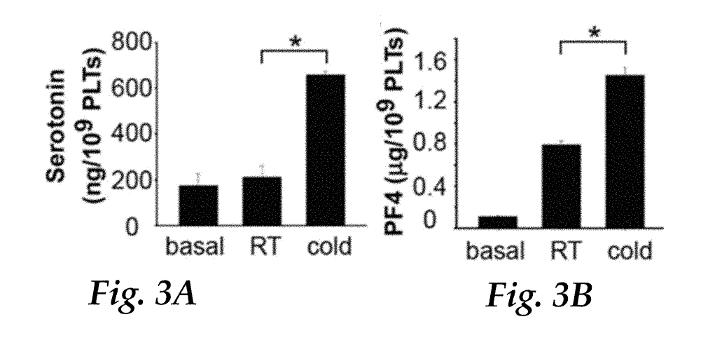

The present disclosure shows that cold storage of platelets induces secretion of serotonin from dense granules and secretion of platelet factor 4 (PF4) from alpha granules (FIGS. 3A and 3B).

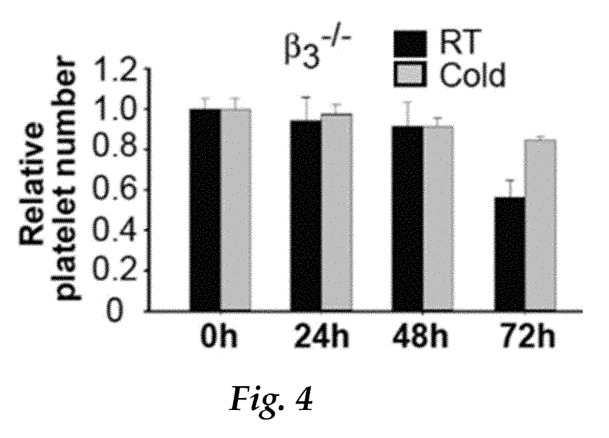

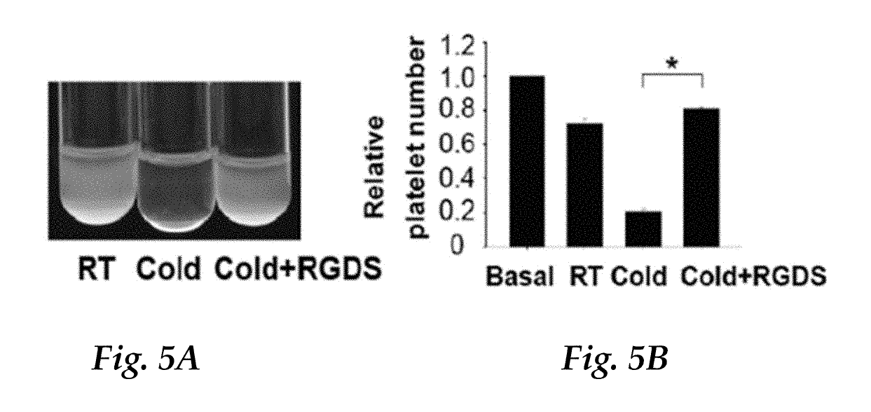

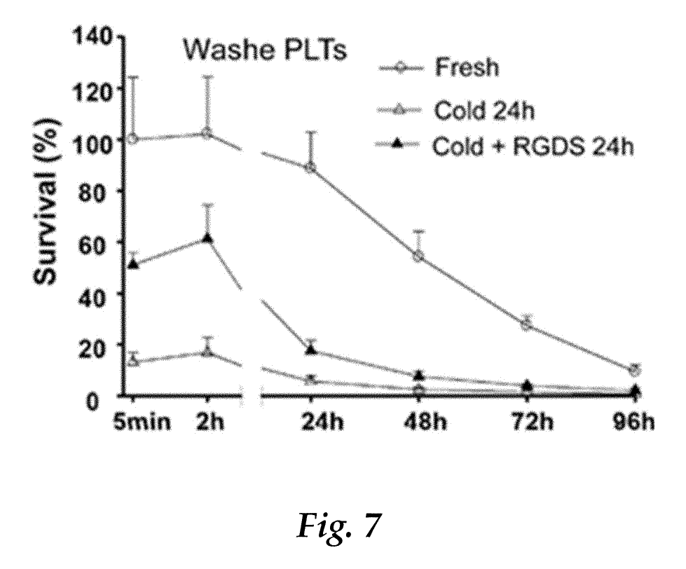

Moreover, the present disclosure shows that cold temperature induces integrin-dependent platelet activation and aggregation. Platelets deficient in integrin beta-3 stored in the cold exhibited no visible sign of aggregation and reduction in platelet counts (FIG. 4). Further, the RGDS peptide, an integrin inhibitor, inhibited cold-induced aggregation of platelets and reduction in platelet counts (FIGS. 5A and 5B). Additionally, the RGDS peptide inhibited partial clearance of cold-stored platelet demonstrating that platelet activation and aggregation contribute to cold-induced rapid clearance (FIG. 7). Because transfused platelets need to stop bleeding efficiently, inhibition of cold-induced platelet activation must be reversible. Integrin is required for platelet hemostatic function. Therefore, integrin inhibitors are unlikely to be developed as reagents for cold storage.

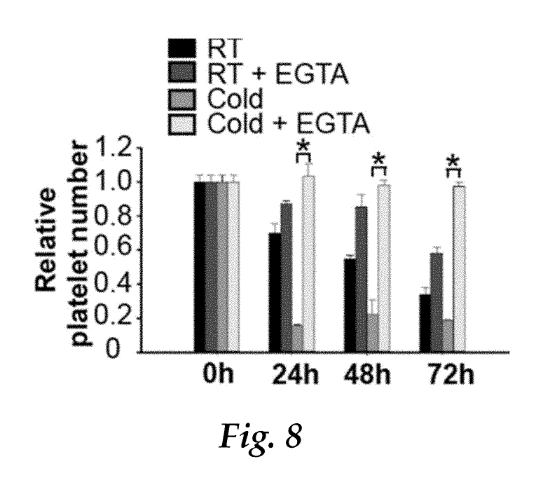

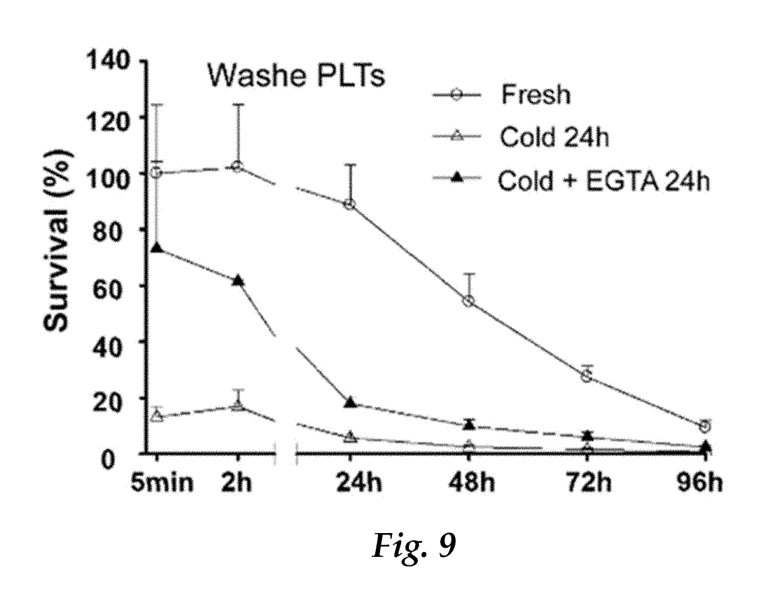

In the search for other reagents, it was found that calcium (Ca⁺⁺) binding to the extracellular membrane of platelets is required for cold-induced platelet activation. The present disclosure shows that ethylene glycol-bis(β-aminoethyl ether)-N,N,N′,N′-tetraacetic acid (EGTA), a cell impermeable calcium chelator, completely inhibited cold-induced platelet aggregation. Moreover, EGTA treatment inhibited quick clearance after cold storage of washed platelets. About 75% of the cold-stored platelets remained in circulation for more than two hours (FIG. 9). EGTA is also able to inhibit reduction in platelet counts induced by cold storage (FIG. 8).

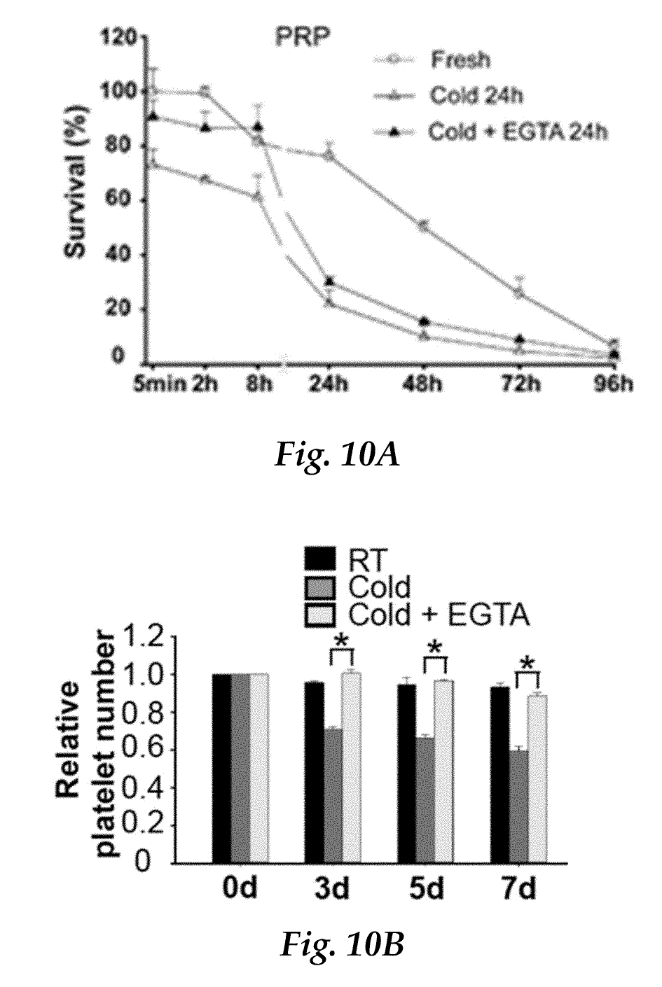

Most platelet products used for transfusion are in plasma. Accordingly, it is important to determine whether plasma behaves similarly as washed platelet. The present disclosure shows that cold storage of platelet-rich plasma (PRP) also resulted in platelet aggregation and reduction in platelet counts, though to a lesser extent (FIG. 10B). This is consistent with the finding that clots are often observed in the cold-stored platelet products and that about 80% of the cold-stored platelet products are discarded after three days of storage. The present disclosure also shows that EGTA inhibited platelet aggregation and maintained platelet counts in PRP. Additionally, the present disclosure shows EGTA inhibited clearance of cold stored platelets in PRP (FIG. 10A)

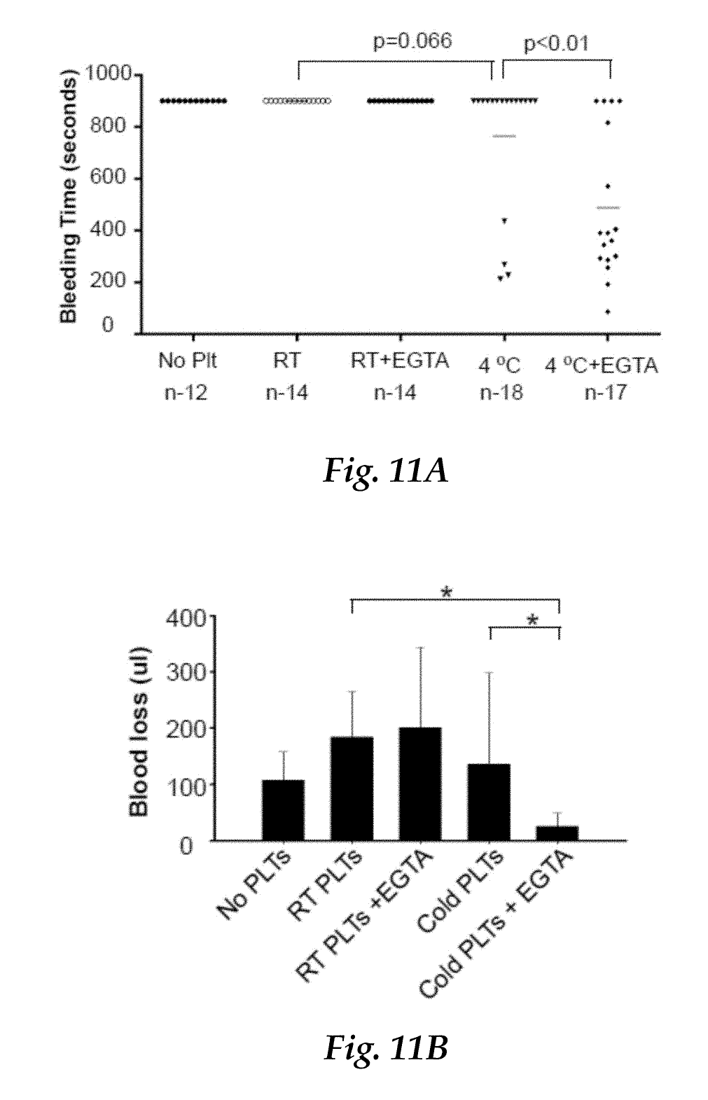

The present disclosure shows that EGTA treated, cold-stored platelets efficiently prevented bleeding in mice. Mice deficient in GP1bα have thrombocytopenia and bleeding tendency. Injection of EGTA-treated, cold-stored PRP into GP1bα deficient mice significantly shortened tail-bleeding times (FIG. 11). The GP1 ba deficient mice without platelet transfusion or that received RT-stored platelets could not stop bleeding (bleed>15 min). In contrast, 75% of the GP1bα deficient mice that received EGTA-treated, cold-stored PRP stopped bleeding.

The present disclosure describes storing in vitro platelets in the cold for longer than three days. In embodiments, the platelets are components of a platelet composition including one or more Ca++ chelators and the platelets. The platelets can be prepared from blood or whole blood as a platelet concentrate or can be in any form that can be stored in the cold.

As an example, the platelet concentrate is in the form of platelet rich plasma (PRP). PRP is obtained by apheresis or from whole blood by centrifugation to remove blood cells. It is a concentrated preparation of platelets in a small volume of plasma. It has a higher concentration of growth factors than whole blood, which is especially useful for wound healing. Platelet counts in PRP is in the range from about 500,000 to about 1,200,000 per cubic millimeter or more. PRP includes unactivated platelets, activated platelets, one or more platelet releasates, or a combination thereof. PRP may be obtained using autologous, allogenic, or pooled sources of platelets and/or plasma. PRP may also be obtained from a variety of mammalian sources, including humans. PRP in the composition can be buffered to physiological pH. In embodiments, platelets can be obtained using the platelet-rich plasma method or the buffy coat method or can be collected by apheresis.

As another example, platelets can be generated in vitro from stem cells such as induced pluripotent stem cells.

In embodiments, the platelet composition includes platelets in any form and one or more Ca⁺⁺ chelators. In particular embodiments, the platelet composition includes PRP and one or more Ca⁺⁺ chelators. The Ca⁺⁺ chelators can be cell permeable or cell impermeable. Examples of cell impermeable or nonpermeable Ca⁺⁺ chelators include ethylenediaminetetraacetic acid (EDTA), ethylene glycol-bis(β-aminoethyl ether)-N,N,N′,N′-tetraacetic acid (EGTA), 1,2-bis(o-aminophenoxy)ethane-N,N,N′,N′-tetraacetic acid (BAPTA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), citric acid, citrate ions, and the cell-permeable isoforms of Ca⁺⁺ chelators. Examples of sources of citrate ions include sodium citrate, potassium citrate, and lithium citrate. Examples of cell permeable isoforms of Ca⁺⁺ chelators include BAPTA-AM, EGTA-AM, EDTA-AM, DTPA-AM, and fructose 1, 6-bisphosphate (FBP).

The platelet compositions described herein can include other excipients suitable for administration to mammalian subjects. As an example, the platelet composition can include isotonic sodium chloride solution, physiological saline, normal saline, dextrose 5% in water, dextrose 10% in water, Ringer solution, lactated Ringer solution, Ringer lactate, Ringer lactate solution, modified Ringer solution with reduced calcium chloride, and platelet additive solution including commercial platelet additive solution. The Ringer solution includes sodium chloride, potassium chloride, calcium chloride, sodium bicarbonate, and minerals such as magnesium chloride, dissolved in distilled water. Other excipients may include crystalloids and colloids. As an example, the solution may include 3.3% dextrose/0.3% saline, 5% dextrose, normal saline, or gelofusine.

The platelet composition can include other therapeutic agents. Examples of therapeutic agents include growth factors, growth inhibitors, coagulation factors, albumin, immunoglobulin, cytokines, enzymes, and lipid or phospholipid.

The platelet composition described herein can be a pharmaceutical composition for administering to mammalian subjects, for example, humans. In such a pharmaceutical composition, all the components are suitable for administering to mammalian subjects. The components are pharmaceutically acceptable components.

The present disclosure describes a novel method of storing platelets in the cold which includes adding to a platelet composition one or more Ca⁺⁺ chelators to a concentration from about 1 μM to about 100 mM in the composition. In embodiments, the concentration of Ca⁺⁺ chelators in the platelet composition is about 1 μM to about 75 mM, about 1 μM to about 50 mM, about 1 μM to about 15 mM, about 1 μM to about 10 mM, about 1 μM to about 5 mM, about 1 μM to about 2.5 mM, about 1 μM to about 1 mM, about 1 μM to about 750 μM, or about 1 μM to about 500 μM.

In embodiments, the method includes adding one or more Ca⁺⁺ chelators to a platelet composition including PRP and storing the platelet composition in the cold. Cold storage can be at a temperature from above 00° C. to any temperature below 22° C. Lowering the temperature reduces the growth of bacteria. In embodiments, the platelet composition is stored at about 1° C. to about 20° C., at about 1° C. to about 18° C., at about 10° C. to about 14° C., at about 10° C. to about 10° C., at about 10° C. to about 8° C., or at about 1° C. to about 5° C. In particular embodiments, the platelet composition is stored at about 4° C.

The method described herein enables the storage of the platelet compositions in the cold for at least three days. The method enables the storage of the platelet composition in the cold for about three days to about 15 days, about four days, about five days, about six days, about seven days, about eight days, about nine days, about 10 days, about 11 days, about 12 days, about 13 days, about 14 days, or about 15.

The platelet compositions described herein or prepared by the method described herein not only can be stored in the cold for longer than three days but also can be used without testing for bacteria or other undesirable microorganisms prior to use because storage in the cold inhibits growth of microorganisms.

The platelet compositions described herein are useful for preventing and treating platelet diseases and disorders. The principle function of platelets is to promote hemostasis which is the process of stopping bleeding. Platelet disorder can be caused by a deficient number of platelets, dysfunctional platelets, or an excessive number of platelets. Low platelet counts can cause spontaneous or excess bleeding. As an example, the platelet compositions described herein can be administered to a subject to increase the platelet count. As another example, the platelet compositions can be administered to a subject when the subject's platelets are characterized by abnormal morphology and/or abnormal function.

Platelets play a role in the regulation of wound healing and inflammation. Platelet therapy has been recognized as a treatment for injury, wound, sepsis, etc. The platelet compositions described herein, which is stored in the cold, can treat the subjects without bleeding. The platelet compositions described herein can be injected into veins or in any parts of the body in subjects to treat injuries including sepsis. The platelet composition described herein can promote wound healing and regulate inflammation including arthritis. The platelet composition can be used also to treat subjects with sports injury including knee injuries and joint injuries.

In embodiments, the platelet compositions described herein are useful for preventing and treating bleeding disorders, for example, thrombocytopenia and traumatic hemorrhages. The platelet composition described herein, which is stored in the cold, can treat bleeding in subjects in reduced time as compared to a platelet composition stored at RT. The platelet composition described herein can reduce the bleeding time significantly. In other embodiments, the platelet composition can be used to reduce blood loss by about 10% to about 85%, at least 10%, at least 20%, at least 25%, at least 30%, at least 35%, at least 40%, at least 45%, at least 50%, at least 55%, at least 60%, at least about 65%, at least 70%, at least 75%, at least 80%, or about 85%.

The platelet compositions described herein are administered to the subject by injection or by transfusion.

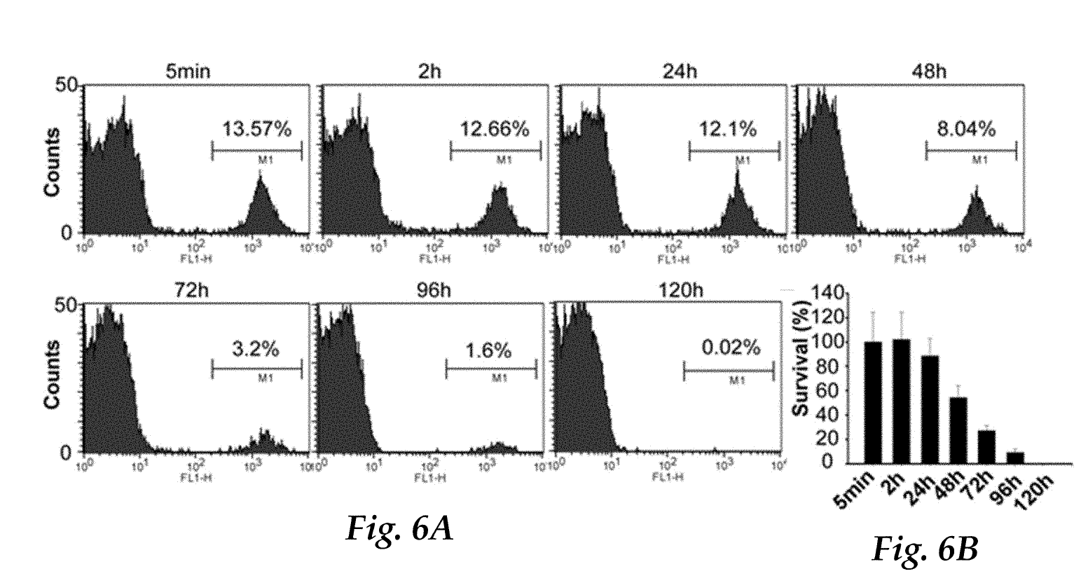

The present disclosure provides a novel method for monitoring platelet clearance and/or life span of transfused platelets in circulation. Conventional methods of labeling platelets with radioactive material or fluorescent dye to differentiate transfused platelets from the platelets of the recipient may activate platelets or change the structure of the platelets and affect the life span of the platelets after transfusion. The method described herein involves using platelets from GFP (Green Fluorescent Protein) mice (C57BL/6-Tg(CAGEGFP)1 Osb/J mice from Jackson Laboratories). The life span of platelets prepared from GFP mice in recipient mice is four to five days which is consistent with previous findings (FIG. 6B). In embodiments, the method for monitoring platelets in circulation described herein includes obtaining fresh platelets from GFP mice, preparing the platelets for injection, injecting the platelet into recipient mice, collecting blood from recipient mice, and monitoring and/or detecting the transfused platelets in circulation. The transfused platelets can be monitored and/or detected by various techniques including flow cytometry and intravenous microscope technology. Using the method described herein, the transfused platelets in the recipient mice can also be quantitated. Accordingly, the method can be used to determine the life span of transfused platelets in vivo and to monitor the clearance of the transfused platelets. In embodiments, the method is a flow cytometry-based assay.

The method can be performed with any transgenic animal that has platelets that express a marker. The marker can be any detectable marker, such as a fluorescent marker. In embodiments, the marker is GFP. The platelets from the transgenic animal can be injected into a recipient animal of the same species. In embodiments, the transgenic animal is a mouse and the recipient animal is a mouse.

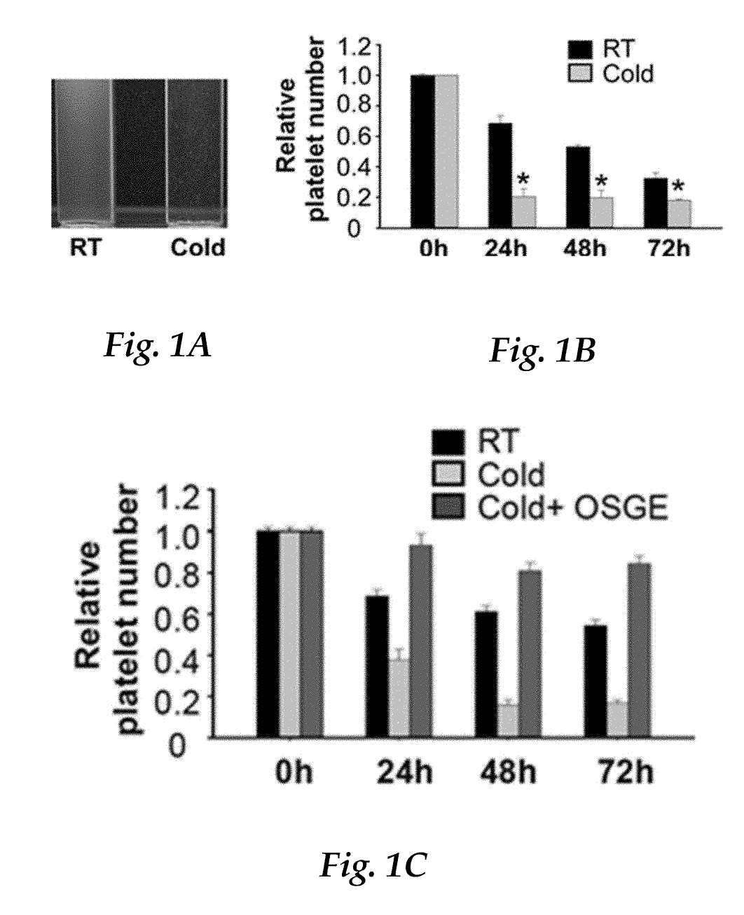

It is known that storage of platelets in the cold induces rapid clearance after transfusion into a subject. The present disclosure shows that aggregation of cold-stored platelets is observed at 24 hours (FIG. 1A). Moreover, at 24 hours, the number of platelets decreased to about 20% of the total number of platelets at 0 hour, the starting point (FIG. 1B). Additionally, for platelets stored at RT, the total number of platelets also decreased with time.

Further, the present disclosure shows that removing the extracellular domain of GPIbα by pretreatment of platelets with O-sialoglycoprotein endopeptidase inhibited platelet aggregation and reduction in platelet numbers (FIG. 1C). The present disclosure shows that O-sialoglycoprotein endopeptidase can be used to inhibit aggregation of platelets stored in the cold.

The present disclosure describes methods of inhibiting rapid clearance of platelets after cold storage. One method involves inhibiting the activation of integrin by adding an integrin inhibitor to a platelet composition prior to cold storage. Another method involves inhibiting the binding of fibrinogen to the platelet by adding a fibrinogen inhibitor to the platelet composition prior to cold storage. Inhibitors of integrins are well-known. Examples of such inhibitors include the RGDS peptide and other molecules such as abciximab, eptifibatide, tirofiban, rexiban, and orbofiban. A third method involves using O-sialoglycoprotein endopeptidase. Although these inhibitors and endopeptidase can be used to prevent rapid clearance of platelets after cold storage, they cannot be used with platelet compositions that are to be administered to a subject that is in need of treatment because these inhibitors and endopeptidase irreversibly inhibit platelet activation and will continue to inhibit platelet activation. As explained above, inhibition of cold-induced platelet activation must be reversible so that the platelets administered to a subject would be functional. Alternatively, one way of using these inhibitors and/or endopeptidase is to remove them from the platelets or neutralize or deactivate them completely before administering them to a subject.

In embodiments, the method of inhibiting in vivo or in vitro rapid clearance of platelets after cold storage involves adding one or more Ca⁺⁺ chelators to the platelet composition prior to cold storage. The platelet composition including the one or more Ca⁺⁺ chelators can be administered to a subject in need of treatment, such as in need of increasing platelet counts for blood clotting to stop bleeding.

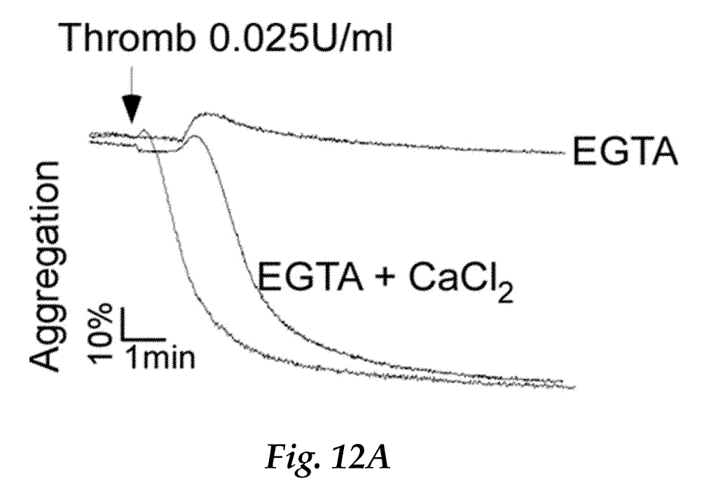

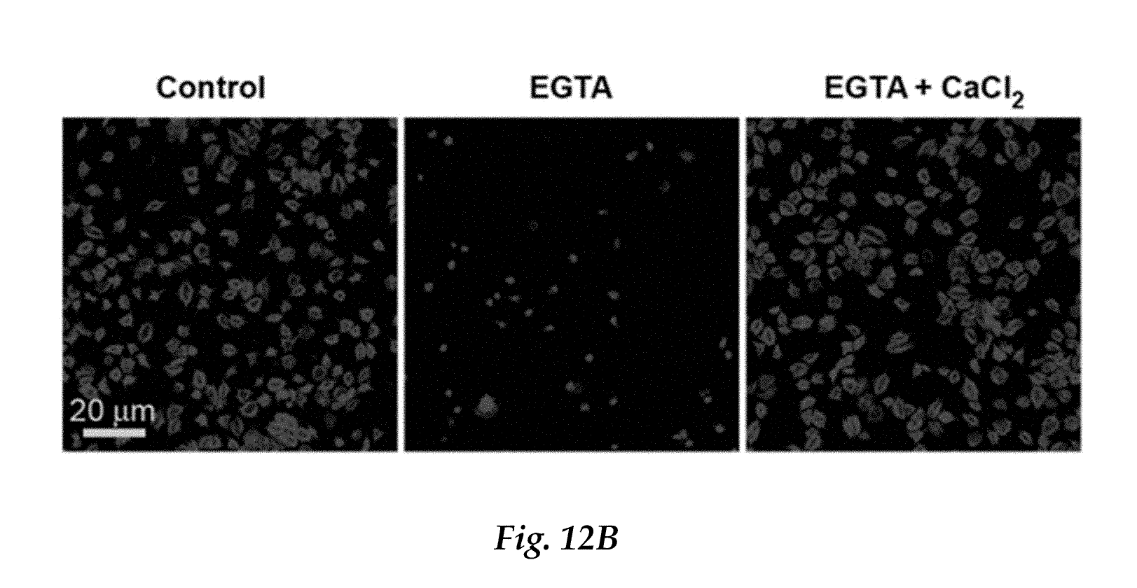

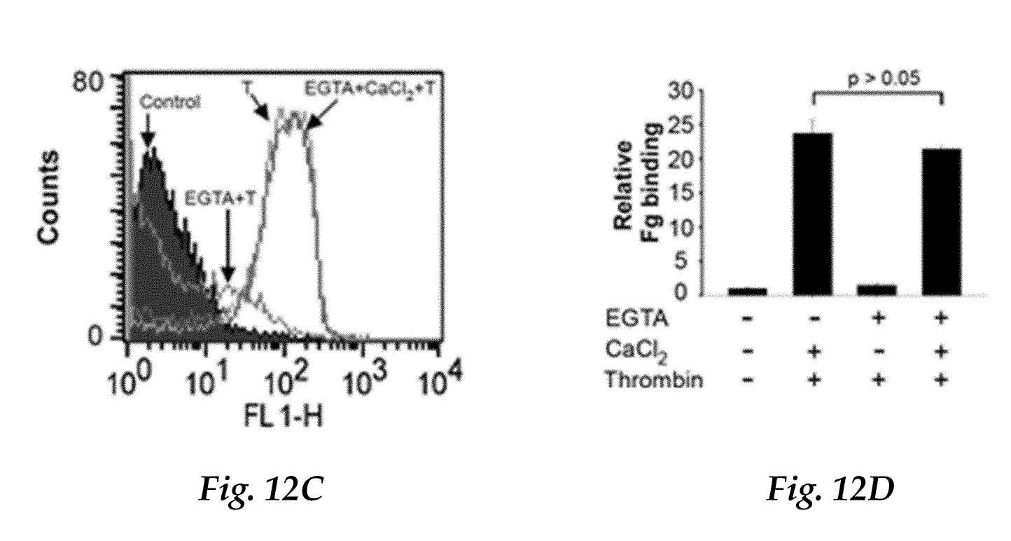

The present disclosure shows that EGTA inhibited platelet spreading on fibrinogen is reversible. Platelets treated with EGTA inhibited platelet adhesion and spreading on fibrinogen which was reversed by adding physiological concentration of CaCl₂) (FIG. 12B). Moreover, thrombin induced fibrinogen binding to platelets was inhibited by EGTA and was reversed by physiological concentration of CaCl₂) (FIG. 12C). The present disclosure describes a method of reversing the action of Ca⁺⁺ chelators on platelets by adding physiological concentration of Ca⁺⁺ to the platelets.

The present disclosure also describes maintaining platelet counts of a platelet composition during storage in the cold. The method involves inhibiting the activation of integrin or inhibiting the binding of fibrinogen to the platelet. As described above, such inhibitors are well-known, but they are not the best candidates for storage of platelets in the cold, if the platelet composition is to be administered to a subject in need of treatment.

In contrast, for a platelet composition that is to be administered to a subject in need of treatment, one or more Ca⁺⁺ chelators can be added to a platelet composition prior to storage to maintain platelet counts of the platelet composition during storage. The storage can be in the cold or at RT.

It is known that RT storage of platelets results in modification of platelet morphology and function, also known as platelet lesions. The present disclosure shows that platelets stored at RT for two days or longer are not able to respond to agonist stimulation. The present disclosure describes a method of extending the shelf-life of platelets by storing them in the cold and by the addition of Ca⁺⁺ chelators to the platelets prior to storing them in the cold. The shelf-life of the platelets stored in the cold will be from about one to about 10 days longer as compared to the platelets stored at RT. The present disclosure also describes a method of extending the shelf-life of platelets stored at RT by adding one or more Ca⁺⁺ chelators to protect them from platelet lesions and to help them maintain their function and morphology. The shelf-life of the platelets stored at RT with added Ca⁺⁺ chelators is at least four days, at least five days, at least six days, at least seven days, at least eight days, at least nine days, at least 10 days, at least 11 days, at least 12 days, at least 13, at least 14 days, or 15 days. Although Ca⁺⁺ chelators can extend the shelf-life of platelets stored at RT, storage of platelets at RT is not preferred due to the potential growth of bacteria and microorganisms.

The method of storing platelets in the cold described herein maintains the hemostatic function of the platelets. The method includes adding one or more Ca⁺⁺ chelators to a platelet composition and storing the composition in the cold for about three to 15 days. The platelet composition maintains its hemostatic function after storage in the cold better than platelet composition stored at RT. As an example, the EGTA treated platelets stored in the cold maintained its ability to respond to ADP and the PAR1 peptide better than platelets stored at RT.

Platelet activation involves multiple pathways including the PI3K/Akt pathway, the MAP kinase pathway, and the Src kinase pathway. The present disclosure shows that cold temperature induces Src kinase phosphorylation.

Methods disclosed herein include treating mammalian subjects. Examples of mammalian subjects include humans, dogs, cats, horses, cow, pigs, mouse, and rats. Subjects in need of a treatment (in need thereof or in need of platelets or platelet function) are subjects having platelet diseases or disorders.

Platelet diseases and disorders include conditions characterized by low platelet counts, dysfunctional platelets, and excess number of platelets. Normal platelet count is about 150,000 to 350,000 per microliter of blood. Low platelet count is less than 50,000 per microliter of blood. Low platelet count can cause excess bleeding, while high platelet counts (over one million per microliter of blood) can cause excessive blood clotting. In embodiments, the platelet compositions described herein are for treating subjects with low platelet counts and/or abnormal and/or dysfunctional platelets.

The present disclosure also provides kits for preparing platelets for storage in the cold or at RT. The kits include a container for collecting platelets and one or more Ca⁺⁺ chelators. The kit may include excipients including Ringer solution and the like for adding to the platelets.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As an example, lack of a material effect is evidenced by lack of a statistically-significant ability of the embodiment to improve the platelet count or platelet function in vitro or in vivo.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; +19% of the stated value; ±18% of the stated value; +17% of the stated value; +16% of the stated value; +15% of the stated value; +14% of the stated value; ±13% of the stated value; ±12% of the stated value; +11% of the stated value; +10% of the stated value; +9% of the stated value; +8% of the stated value; +7% of the stated value; +6% of the stated value; +5% of the stated value; ±4% of the stated value; +3% of the stated value; +2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXEMPLARY EMBODIMENTS

The following are exemplary embodiments:

1. An in vitro platelet composition including platelets and one or more Ca⁺⁺ chelators.
2. The platelet composition of embodiment 1, wherein the platelets are in a concentrated form.
3. The platelet composition of embodiment 1 or 2, wherein the concentrated form includes platelet rich plasma (PRP).
4. The platelet composition of any one of embodiments 1-3, wherein the PRP is prepared from blood.
5. The platelet composition of any one of embodiments 1-4, wherein the blood includes mammalian blood.
6. The platelet composition of any one of embodiments 1-5, wherein the mammalian blood includes human blood.
7. The platelet composition of any one of embodiments 1-6, wherein the one or more Ca⁺⁺ chelators are cell impermeable or cell permeable Ca⁺⁺ chelators.
8. The platelet composition of any one of embodiments 1-7, wherein the one or more Ca⁺⁺ chelators are ethylenediaminetetraacetic acid (EDTA), ethylene glycol-bis(β-aminoethyl ether)-N,N,N′,N′-tetraacetic acid (EGTA), fructose 1, 6-bisphosphate (FBP), 1,2-bis(o-aminophenoxy)ethane-N,N,N′,N′-tetraacetic acid (BAPTA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), citric acid, or citrate ions.
9. The platelet composition of any one of embodiments 1-8, wherein the source of citrate ion is sodium citrate, potassium citrate, or lithium citrate.
10. A method of storing the platelet composition of any one of embodiments 1-9, wherein the method includes storing the composition at a temperature above 0° C. and less than 22° C.
11. A method of storing the platelet composition of any one of embodiments 1-9, wherein the method includes storing the composition at room temperature.
12. A method of preparing and storing a platelet composition, wherein the method includes isolating blood from a subject, preparing a platelet composition using the isolated blood, adding one or more Ca⁺⁺ chelators to the platelet composition, and storing the platelet composition at a temperature above 0° C. and less than 22° C.
13. A method of inhibiting platelet aggregation during and after cold storage, wherein the method includes adding one or more Ca⁺⁺ chelators to a platelet composition and storing the platelet composition at a temperature above 0° C. and less than 22° C.
14. A method of preventing rapid clearance of platelets after cold storage, wherein the method includes adding one or more Ca⁺⁺ chelators to a platelet composition and storing the platelet composition at a temperature above 0° C. and less than 22° C.
15. A method of maintaining platelet count of a platelet composition during cold storage, wherein the method includes adding one or more Ca⁺⁺ chelators to a platelet composition and storing the platelet composition at a temperature above 0° C. and less than about 22° C.
16. A method of inhibiting platelet aggregation or preventing rapid clearance of platelets after cold storage, wherein the method includes adding one or more integrin inhibitors, one or more fibrinogen inhibitors, or an O-sialoglycoprotein endopeptidase to a platelet composition and storing the platelet composition at a temperature above 0° C. and less than 22° C.
17. A method of maintaining platelet count of a platelet composition during cold storage, wherein the method includes adding one or more integrin inhibitors or fibrinogen inhibitors to a platelet composition and storing the platelet composition at a temperature above 0° C. and less than 22° C.
18. The method of any one of embodiments 10-17, wherein the blood includes mammalian blood.
19. The method of any one of embodiments 10-18, wherein the mammalian blood includes human blood.
20. The method of any one of embodiments 10-19, wherein the one or more Ca⁺⁺ chelators are cell permeable or cell impermeable Ca⁺⁺ chelators.
21. The method of any one of embodiments 10-20, wherein the one or more Ca⁺⁺ chelators are ethylenediaminetetraacetic acid (EDTA), ethylene glycol-bis(p-aminoethyl ether)-N,N,N′,N′-tetraacetic acid (EGTA), fructose 1, 6-bisphosphate (FBP), or 1,2-bis(o-aminophenoxy)ethane-N,N,N′,N′-tetraacetic acid (BAPTA).
22. The method of embodiment 16 or 17, wherein the integrin inhibitor is RGDS peptide.
23. The method of any one of embodiments 10-17, wherein the platelet composition is stored in the cold for five to 15 days, at least five days, at least seven days, at least 9 days, at least 11 days, at least 13 days, or 15 days.
24. The method of any one of embodiments 10-23, wherein the platelet composition is stored at between 0° C. to 14° C., less than 12° C., less than 9° C., less than 7° C., less than 5° C., or at 4° C.
25. A method of treating a subject in need of platelet transfusion, wherein the method includes transfusing the subject with the platelet composition of any one of embodiments 1-9.
26. The method of embodiment 25, wherein the subject has low platelet count, abnormal platelet morphology, or abnormal platelet function.
27. The method of any one of embodiments 25 or 26, wherein the subject has thrombocytopenia or a sport injury.
28. A method of inhibiting bleeding in a subject, wherein the method includes administering to the subject the composition of any one of embodiments 1-9.
29. The method of embodiment 28, wherein the blood loss is reduced by about 25%, 30%, 45%, 50%, 55%, 60%, 65%, 70%, 80% or 85% as compared to blood loss using RT-stored platelet without added Ca⁺⁺ chelators.
30. A method for monitoring transfused platelets in circulation, wherein the method includes obtaining platelets from a transgenic animal that expresses a marker, injecting the platelets into a recipient animal, and monitoring and/or detecting the transfused platelets in circulation.
31. The method of embodiment 30, wherein the recipient animal is of the same species as the transgenic animal.
32. The method of embodiment 30 or 31, wherein the marker is GFP.
33. The method of any one of embodiments 30-32, wherein the transgenic animal is a GFP mouse and the recipient animal is a mouse.
34. The method of any one of embodiments 30-33, wherein the method further includes collecting blood from the recipient animal for monitoring and/or detecting transfused platelets in circulation.
35. The method of any one of embodiments 30-34, wherein the monitoring and/or detecting is performed using flow cytometry.
36. The method of any one of embodiments 30-35, wherein the method monitors life span and/or clearance of the platelet in circulation.
37. A method of extending the shelf-life of a platelet composition, the method includes adding one or more Ca⁺⁺ chelators to the platelet composition, and storing the platelet composition in the cold or at room temperature.
38. The method of embodiment 37, wherein the shelf-life of the platelet composition is four to 15 days.
39. A method of inducing Src kinase phosphorylation of platelets, wherein the method includes storing the platelets in the cold.
40. A method of reversing the function of Ca⁺⁺ chelators on cold stored platelets, wherein the method includes adding physiological concentration of calcium to the platelets.
41. The method of embodiment 40, wherein the function of Ca⁺⁺ chelators is inhibiting platelet spreading on fibrinogen or inhibiting integrin ligand binding.
42. The method of embodiment 40 or 41, wherein the calcium added is in the form of calcium chloride.
43. A method of maintaining hemostatic function of platelets, wherein the method includes adding one or more Ca⁺⁺ chelators to the platelet and storing the platelet in the cold.
44. The method of embodiment 43, wherein the platelets are in a platelet composition.
45. The method of embodiment 43 or 44, wherein the platelet composition includes PRP.

### Examples

### Introduction

Cold-stored platelets are cleared either by Kuffer cells in liver due to exposure of the N-acetyl-glucosamine terminals on the glycoprotein Ibα (GPIbα) or by hepatocytes due to exposure of the galactose terminal glycans on GPIbα. Unfortunately, a phase 1 clinical trial testing the idea of restoring survival of chilled platelets by adding uridine 5′-diphosphogalactose to block the explored N-acetyl-glucosamine terminal failed, indicating that increased clearance after cold storage is not solely due to deglycosylation of GPIbα. The mechanism by which cold-stored platelets are cleared are not known. The following examples identifies the molecular mechanism that triggers platelet activation and clearance during cold storage and provides a novel process for storing platelet in the cold.

### Example 1A. Cold Storage Induced Platelet Aggregation

To investigate the mechanism of the rapid clearance after cold storage, washed platelets from C57BL/6 mice were suspended in Ca⁺⁺ free Tyrode's solution (12 mM NaHCO₃, 138 mM NaCl, 5.5 mM glucose, 2.9 mM KCl, 2 mM MgCl₂, 0.42 mM NaH₂PO₄, 10 mM HEPES, pH 7.4). and stored at RT or 4° C. Washed platelets from C57BL/6 mice were stored at RT or 4° C. for 24 hours. After storage, platelets were incubated with Oregon Green-labeled fibrinogen at 22° C. for 30 min. Fibrinogen binding to platelets was analyzed by flow cytometry. Surprisingly, aggregates were visible in the cold-stored platelets after 24 hours (FIG. 1A), suggesting that cold storage induced platelet aggregation. To confirm this observation, single platelet count was measured using a HEMAVET HV950FS multispecies hematology analyzer. Indeed, platelet counts were reduced dramatically after platelets were stored at 4° C. for over 24 hours (FIG. 1B). Platelet counts were also gradually reduced when stored at RT.

### Example 1B. O-Sialoglycoprotein Endopeptidase Inhibited Cold-Induced Platelet Aggregation

To determine the molecular mechanism by which cold induces platelet activation, washed platelets prepared from C57BL/6 mice were stored at RT, cold, or cold with O-sialoglycoprotein endopeptidase (OSGE). Pretreating platelets with O-sialoglycoprotein endopeptidase (OSGE) stripped the extracellular domain of GP1bα inhibited platelet aggregation and inhibited the reduction in platelet numbers (FIG. 1C), suggesting that cold-induced platelet activation involves GPIbα. These data are consistent with previous findings that GP1bα plays a key role in the cold-elicited rapid clearance of platelets. GPIbα-dependent platelet activation is one of the major mechanisms leading to rapid clearance of cold-stored platelets.

### Example 2A. Cold-Induced Integrin Activation

Platelet aggregation is a consequence of integrin activation. Thus, fibrinogen binding to platelets was measured to determine whether cold could induce integrin activation. Washed platelets from C57BL/6 mice were stored at RT or 4° C. for 24 hours. After storage, platelets were incubated with Oregon Green-labeled fibrinogen at 22° C. for 30 min. Fibrinogen binding to platelets was analyzed by flow cytometry. As expected, fibrinogen binding to cold-stored platelets was increased, compared with the RT stored platelets (FIGS. 2A and 2B). These data indicate that cold induces integrin activation in platelets.

### Example 2B. Cold Temperature Induced Src Kinase Phosphorylation

Platelet activation involves multiple signaling pathways, including the PI3K/Akt pathway, the MAP kinase pathway and the Src family kinase pathway, leading to phosphorylation of these kinases. Washed platelets prepared from C57BL/6J mice were stored at RT or 4° C. for various lengths of time. Src phosphorylation was measured by Western blot as described previously. It was found that Src phosphorylation was dramatically increased after cold storage (FIG. 2C). These data suggest that cold storage indeed induces signaling, which may lead to platelet activation.

### Example 3. Cold-Induced Platelet Secretion

Platelet activation is often accompanied by secretion. To verify that cold storage elicits platelet activation, platelet secretion during cold storage was measured. Washed platelets from C57BL/6 mice were stored at RT or 4° C. for 24 hours. Secretion from dense granules was evaluated by measuring serotonin (FIG. 3A) in supernatant and secretion from alpha granules was determined by measuring platelet factor 4 (PF4, FIG. 3B) in supernatant. As shown in FIG. 3, cold storage indeed elicited significant secretion from both dense and alpha granules.

### Example 4. Cold-Induced Platelet Activation was Abolished in the Integrin 33 Deficient Platelets

To determine whether cold-induced aggregation is due to integrin activation, platelet aggregation and platelet counts of the β3 deficient platelets during cold storage were examined. Washed platelets from β^{-/-} mice were stored at RT or 4° C. for various time. β3 deficient washed platelets maintained their platelet counts after being stored at 4° C. even for 72 hours (FIG. 4) and no visible aggregates were observed (data not shown). These data further demonstrate that cold induces integrin-dependent platelet activation and aggregation.

### Example 5. an Integrin Inhibitor RGDS Peptide Inhibited Cold-Induced Platelet Aggregation and Reduction in Platelet Counts

If cold-induced aggregation is due to integrin activation, integrin inhibitors should be able to inhibit this process. Washed platelets from C57BL/6 mice, suspended in Ca⁺⁺ free Tyrode's solution, were added with 1 mM RGDS and stored at 4° C. for 24 hours. Indeed, an integrin inhibitor RGDS peptide inhibited cold-elicited aggregation (FIG. 5A) and reduction in platelet counts (FIG. 5B).

### Example 6. a Novel Assay for Monitoring Platelet Clearance

To monitor the life span of transfused platelets in circulation, a method that can easily differentiate the transfused platelets from recipient platelets was developed. Platelets from C57BL/6-Tg(CAGEGFP)1 Osb/J mice (GFP mice) (From Jackson Laboratories) are GFP positive that are distinct from the recipient platelets by flow cytometry assay (FIG. 6A). C57BL/6J mice were injected retro-orbitally with washed GFP platelets (2.5×10⁸ per mouse). 60 μl blood was collected from mice and PRP was analyzed by flow cytometry. Quantitative results were expressed as percentage of survived transfused platelets (GFP positive cells at 5 min after transfusion was set to 100%; mean±SD; n=5). Using this method, it was found that injection of 2.5×10⁸ platelets to an 18-20 g mouse resulted in ˜13% GFP-positive platelets. When freshly prepared platelets from GFP mice were injected, the life span of the fluorescent platelets in the recipient mice is 4-5 days, consistent with previous findings (FIG. 6B).

### Example 7. RGDS Treatment Reduced Rapid Clearance after Cold Storage

Consistent with previous findings, cold storage resulted in quick clearance (FIG. 7). C57BL/6J mice were injected retro-orbitally with fresh washed GFP platelets or GFP platelets stored at 4° C. for 24 hours (2.5×10⁸ per mouse) with or without 1 mM RGDS. Blood was collected from mice and platelets were analyzed by flow cytometry. Quantitative results were expressed as percentage of survived transfused platelets (GFP positive cells of transfused fresh platelets at 5 min after transfusion was set to 100%, n=4).

More than 80% of the transfused cold-stored platelets were cleared within 5 min after injection. To determine whether platelet activation and aggregations contribute to rapid clearance after cold storage, platelets were pre-treated with RGDS peptide and then stored at 4° C. for 24 hours. About 60% of the RGDS peptide-treated platelets stayed in circulation for more than 2 hours after injection, suggesting that platelet activation and aggregation do contribute to cold-induced rapid clearance. However, the RGDS treatment did not completely resolve the issue with cold storage, and thus there likely exists an additional, integrin activation-independent, mechanism leading to clearance of cold-stored platelets.

### Example 8. Cold-Induced Platelet Activation Required Ca⁺⁺ Binding to the Extracellular Membrane of Platelets

Although integrin inhibitors could inhibit cold-induced platelet activation and efficiently prevent rapid clearance, they cannot be developed as reagents for cold storage of platelets, because transfused platelets need to efficiently stop bleeding, which requires integrin. Therefore, inhibition of cold-induced platelet activation must be reversible. In search of such a method, it was found that a cell-impermeable Ca⁺⁺ chelator, EGTA, completely inhibited cold-induced platelet aggregation and reduction in platelet counts (FIG. 8). Washed platelets from C57BL/6 mice, in the presence or absence of EGTA, were stored at RT or 4° C. for various time. These data suggest that Ca⁺⁺ binding to the extracellular membrane of platelets is required for cold-induced platelet activation.

### Example 9. EGTA Treatment Inhibited Quick Clearance after Cold Storage in Washed Platelets

To determine whether EGTA treatment can prevent cold-elicited rapid clearance, EGTA was added into washed platelets and then incubated at 4° C. for 24 hours. C57BL/6J mice were injected retro-orbitally with fresh washed GFP platelets or GFP platelets stored at 4° C. for 24 hours (2.5×10⁸ per mouse) with or without 50 uM EGTA. Blood was collected from mice and platelets were analyzed by flow cytometry. Quantitative results were expressed as percentage of survived transfused platelets (GFP positive cells of transfused fresh platelets at 5 min after transfusion was set to 100%, n=4). FIG. 9 show that treatment of platelets with EGTA significantly inhibited rapid clearance. About 75% platelets stayed in circulation for more than 2 hours.

### Example 10A. EGTA Treatment Inhibited Quick Clearance by Cold Storage

C57BL/6J mice were injected retro-orbitally with fresh or cold-stored PRP (2.5×10⁸ per mouse) from C57BL/6-Tg(CAGEGFP)1 Osb/J. Blood was collected from the recipients and platelets were analyzed by flow cytometry, n=4). Rapid clearance of cold-stored platelets in PRP is less severe than washed platelets. Inclusion of EGTA significantly reduced quick clearance (FIG. 10A), similar to that observed in washed platelets.

### Example 10B. EGTA Inhibited Platelet Aggregation and Reduction in Platelet Counts in Platelet-Rich Plasma (PRP)

The above data demonstrate that platelet aggregation is one of the major causes of quick clearance after transfusion. This conclusion is drawn based on the experiments using washed platelets. Although a small portion of platelet products uses platelet additive solution (PAS), most platelet products used for transfusion are in plasma. Therefore, whether PRP has a similar phenotype as observed in washed platelets was examined. PRP from C57BL/6 mice, in the presence or absence of EGTA, were stored at RT or 4° C. for various time. It was found that cold storage also resulted in platelet aggregation and reduction in platelet counts, although at a much less extent (FIG. 10B). These data are consistent with previous findings that clots were often observed in the cold-stored platelet products, which contributes to wastage of ˜80% platelet products. Addition of EGTA effectively inhibited reduction in platelet counts by cold storage (FIG. 10B)

### Example 11. EGTA-Treated, Cold-Stored Platelets Efficiently Prevented Bleeding in Mice

To determine whether EGTA-treated, cold-stored platelets can preserve hemostatic function, EGTA-treated PRP was stored at 4° C. for 48 hours and then transfused into GP1bα deficient mice. About 7 to 8 weeks mice (both males and females, sexmatched) deficient in GP1bα received vehicle, RT stored platelets, or cold-stored platelets (1×10⁹ platelets in 0.2 ml PRP per mouse by retro-orbital injection), in the presence or absence of EGTA. After 2 hours, the mice were anesthetized by inhalation of 2-5% isoflurane in 100% oxygen using a vaporizer. The distal portion of the tail (1 mm) was amputated with a scalpel, and the tail was immersed in 12 ml of 0.15 M NaCl at 37° C. Time to stable cessation of the bleeding was defined as the time where no rebleeding for longer than 2 minutes was recorded. To measure blood loss volume, any blood collected from tail transection was frozen at −80° C. overnight. After thawing the following day, 12 ml of deionized water was added to further induce hemolysis. Aliquots of each sample were analyzed via spectrophotometry (SpectraMax Plus384; Molecular Devices, Sunnyvale, Calif.) and diluted further (1:5, 1:10, or 1:20) if necessary. The resulting OD490 nm values (% T) were compared against a standard curve to estimate the blood volume lost.

GPIb-IX complex is essential for platelet production and hemostasis. GP1bα deficiency or loss of function leads to Bernard-Soulier syndrome (BSS). Similar to BSS patients, mice deficient in GP1bα have thrombocytopenia, giant platelets, and bleeding tendency. Injection of EGTA-treated, cold-stored platelets (1×10⁹ platelets per mouse) in PRP into GP1bα deficient mice significantly shortened tail-bleeding times (FIG. 11A) and reduced blood loss (FIG. 11B). In contrast, injection of the same number of RT-stored platelets in the presence or absence of EGTA did not significantly prevent blood loss or reduce tail-bleeding times in the GP1bα deficient mice.

### Example 12A. EGTA Reversibly Inhibited Platelet Aggregation

Washed platelets from C57BL/6J mice suspended in Tyrode's solution were pretreated with EGTA (100 μM). Platelets were then added with buffer or 1 mM CaCl₂). Washed platelets suspended in Tyrode's solution with 1 mM CaCl₂ were used as a control. Platelet aggregation was induced by addition of thrombin (0.025 U/ml).

Incubation of platelets with EGTA at RT inhibited platelet aggregation. However, as shown in FIG. 12, this inhibition is reversible. Addition of physiological dose of Ca⁺⁺ completely reversed this inhibition (FIG. 12). These data suggest that Ca⁺⁺ binding to the extracellular membrane of platelets is required for platelet activation.

To test whether EGTA inhibits platelet function through GPIIb/IIIa, EGTA inhibition of platelet spreading was examined. Treatment of platelets with EGTA inhibited platelet adhesion and spreading on fibrinogen, which was completely reversed by adding 1 mM CaCl₂). These data indicate that Ca⁺⁺ binding to the extracellular domain of GPIIb/IIIa is required for ligand binding, and inhibition of integrin function by EGTA is reversible, and can be completely reversed with a physiological concentration of Ca⁺⁺. These data suggest that Ca⁺⁺ binding to the extracellular membrane of platelets is required for platelet activation and that EGTA inhibits platelet activation not through irreversibly dissociate αIIbβ3 complex.

### Example 12B. EGTA Reversibly Inhibited Platelet Spreading on Fibrinogen

To test whether αIIbβ3 plays a role in EGTA inhibition of platelet function, whether EGTA inhibits platelet spreading on fibrinogen was examined. Washed platelets from C57BL/6J mice suspended in Tyrode's solution were pretreated with EGTA (100 μM). Platelets were then added with buffer or 1 mM CaCl₂). Washed platelets suspended in Tyrode's solution with 1 mM CaCl₂ were used as a control. Platelets were then added to polystyrene dishes coated with 50 μg/mL of fibrinogen and incubated at 37° C. for 60 minutes and labeled with Rhodamine-Phalloidin and photographed using a fluorescence microscope. Treatment of platelets with EGTA inhibited platelet adhesion and spreading on fibrinogen, which was completely reversed by adding 1 mM CaCl₂) (FIG. 12B). These data suggest that Ca⁺⁺ binding to the extracellular domain of αIIbβ3 is required for αIIbβ3 ligand binding function. This conclusion was further confirmed by a fibrinogen binding assay.

### Example 12C. EGTA Reversibly Inhibited Integrin Ligand Binding Function

Washed platelets from C57BL/6J mice suspended in Tyrode's solution were pretreated with EGTA (100 μM). Platelets were then added with buffer or 1 mM CaCl₂). Washed platelets suspended in Tyrode's solution with 1 mM CaCl₂ were used as a control. Platelets were incubated with Oregon Green-labeled fibrinogen and thrombin (0.1 U/ml) at 22° C. for 30 min. Quantitative results were expressed as fibrinogen binding indices (geomean of fluorescence intensity of stimulated platelets/geomean of fluorescence intensity of unstimulated platelets; n=3). Thrombin-induced fibrinogen binding to platelets was completely inhibited by 100 μM EGTA, but was reversed by 1 mM CaCl₂ (FIGS. 12C and 12D). Thus, inhibition of integrin function by EGTA is reversible, which can be completely reversed with the physiological concentration of calcium. Taken together, EGTA inhibition of platelet function involves multiple mechanisms, including cold-elicited integrin activation and integrin ligand binding function.

### Example 13. Platelets Lost their Response to Agonists after RT Storage for Two Days

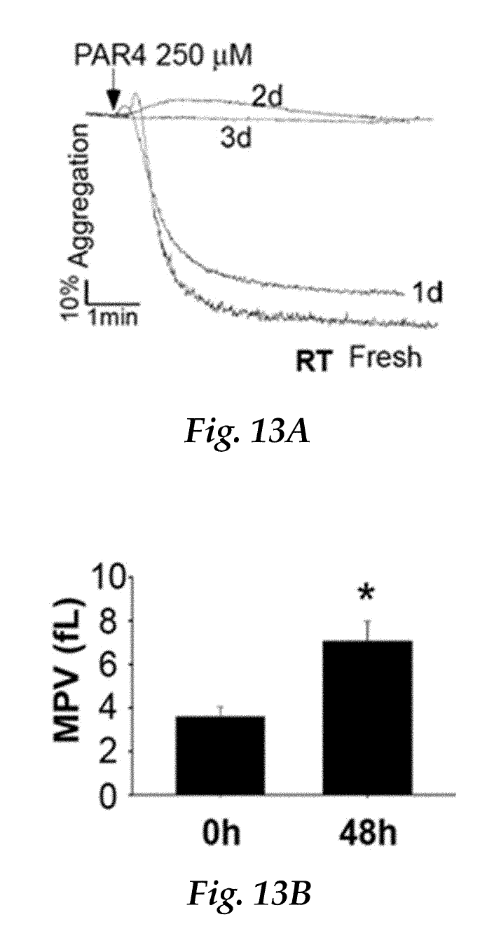

It has been known for several decades that RT storage of platelets causes a series of shape and functional modifications, which are commonly referred to platelet storage lesion. To determine the extent of storage lesion by RT storage, aggregometry was used to examine responses of the RT-stored platelets to agonists. PRP from C57BL/6 mice were stored at RT for various lengths of time. Thrombin receptor agonist PAR4 was added to induce platelet aggregation. PRP from C57BL/6 mice were stored at RT for 48 hours. Platelet size was measured by a HEMAVET HV950FS multispecies hematology analyzer (n=4, p<0.01). Platelet-rich plasma (PRP) stored at RT for two days or longer lost their ability to respond completely to agonist stimulation including the thrombin receptor agonist PAR4 peptide (FIG. 13A) and ADP. RT stored platelets lost normal integrity, because the volume of platelets increased by one-fold after RT storage for two days (FIG. 13B).

### Example 14. Cold Storage Induced Platelet Activation in Human Platelets

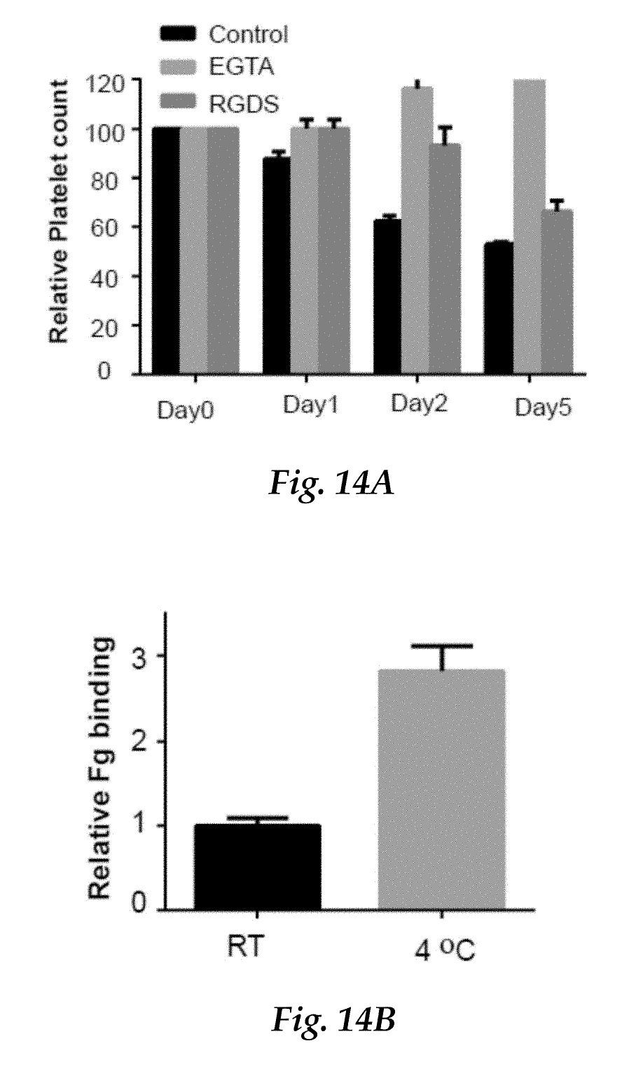

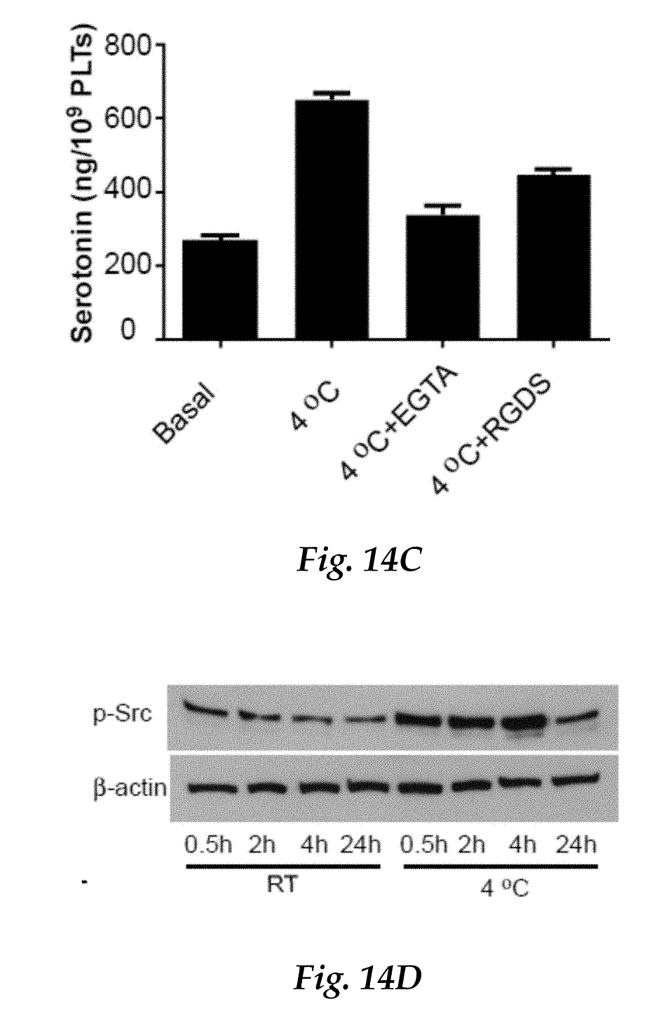

To determine whether cold storage causes aggregation in human platelets, PRP prepared from healthy donors using Anticoagulant Citrate Dextrose Solution A (ACD) as anticoagulant was stored at 4° C., in the absence or presence of EGTA or RGDS peptide. Platelet counts were monitored at various time points. Platelet counts decreased after storage at 4° C. in a time-dependent manner, which was inhibited by EGTA and RGDS (FIG. 14A). Fibrinogen binding to platelets increased after cold storage (FIG. 14B). To determine whether cold storage induces platelet secretion, washed human platelets were pretreated with EGTA or RGDS for 15 min and stored at 4° C. for 24 h. The amount of serotonin secreted in the supernatant was then measured. Cold storage induced platelet secretion of serotonin, but the secretion of serotonin was inhibited by EGTA or RGDS (FIG. 14C). Cold storage also induced Src phosphorylation (FIG. 14D). These data suggest that similar to mouse platelets, human platelets are activated and aggregated during cold storage.

### Example 15. EGTA Treated Human Platelets Stored in the Cold are Better Able to Maintain their Hemostatic Function than Platelets Stored at RT as Shown by their Response to ADP

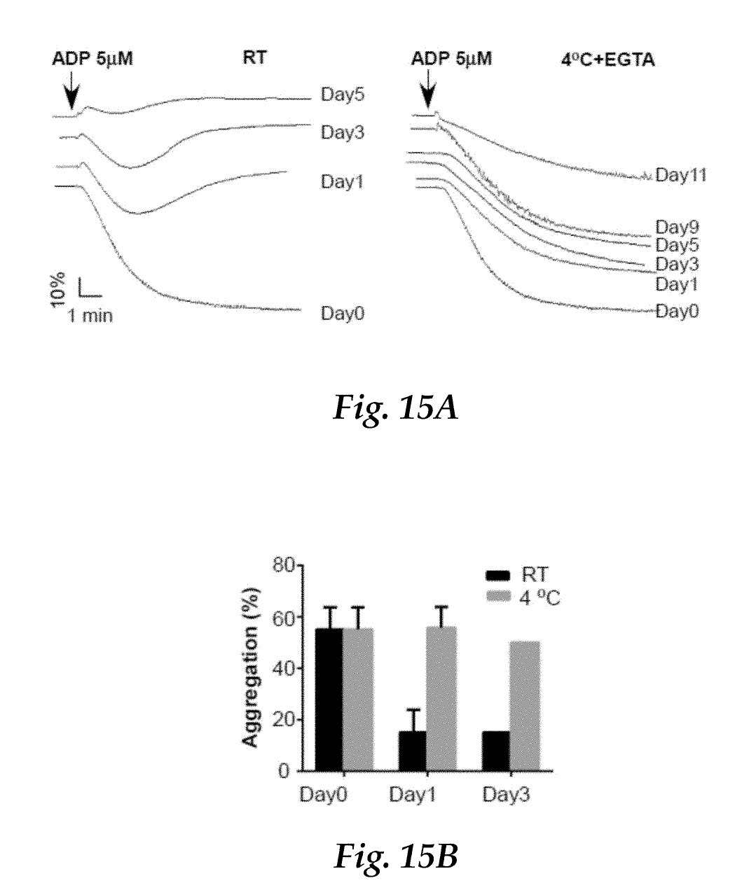

To determine whether EGTA treated platelets stored in the cold can preserve their hemostatic function better than the platelets stored at RT, the abilities of the platelets to respond to ADP were compared. As shown in FIG. 15A, RT-stored platelets lost their ability to respond to ADP after 1 days. In contrast, the EGTA treated platelets stored at 4° C. maintained their ability to respond to ADP even after 9 days. Moreover, as shown in FIG. 15B, the statistical data from three experiments indicate that in the presence of ADP, platelets stored at RT lost their ability to aggregate quickly as compared to EGTA treated platelets stored at 4° C.

### Example 16. EGTA Treated Human Platelets Stored in the Cold are Better Able to Maintain their Hemostatic Function than Platelets Stored at RT as Shown by their Response to the PAR1 Peptide

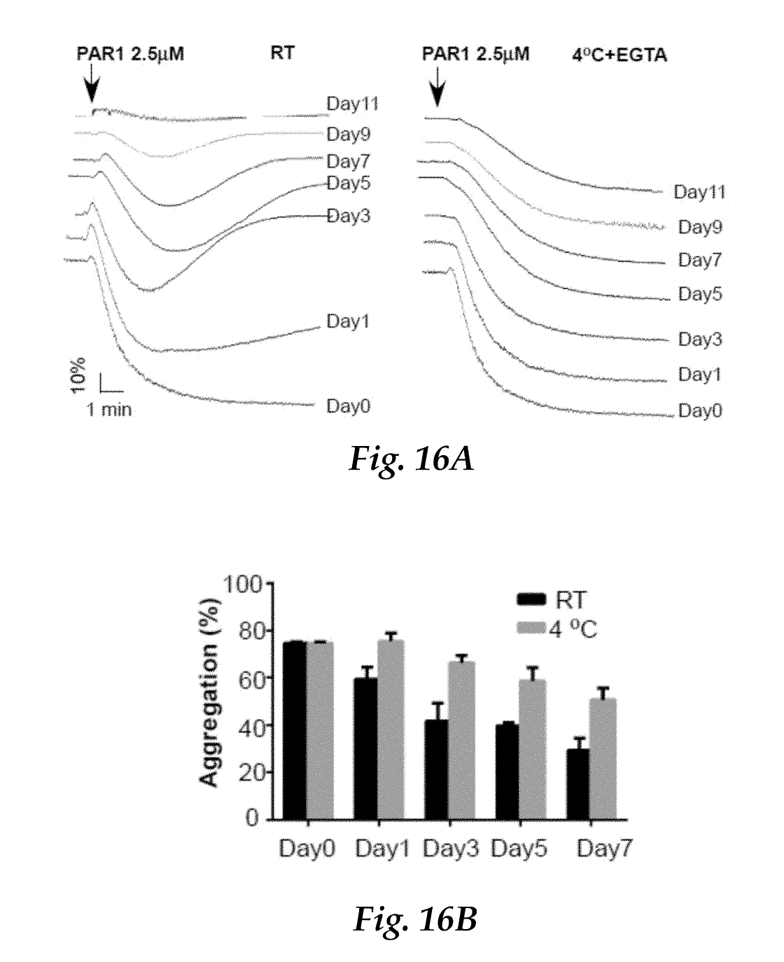

To further determine whether EGTA treated platelets stored in the cold (4° C.) can preserve their hemostatic function better than RT-stored platelets, the abilities of the platelets to respond to the PAR1 peptide were compared. The EGTA-treated platelets stored in the cold maintained their ability to respond to the PAR1 peptide better than the RT stored platelets. As shown in FIG. 16A, RT stored platelets gradually lost their ability to respond to the PAR1 peptide. In contrast, EGTA treated platelets stored at 4° C. maintained their ability to respond PAR1 even after 11 days. Further, as shown in FIG. 16B, the statistical data from three experiments indicate that platelets stored at RT started losing their ability to aggregate in the presence of the PAR1 peptide sooner than EGTA treated platelets stored at 4° C.

In summary, the present disclosure shows: (i) rapid clearance of cold-stored platelets is largely due to integrin activation and aggregation, elicited by Ca⁺⁺ binding to the extracellular domain of platelets; (ii) inclusion of a Ca⁺⁺ chelator in platelets inhibited cold-induced platelet aggregation, significantly reduced rapid clearance, and efficiently prevented bleeding; (iii) there exists an aggregation-independent mechanism for platelet clearance after cold storage; and iv) Ca⁺⁺ chelator helps maintain the hemostatic function of platelets stored in the cold.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The subject matter described above is provided by way of illustration only and should not be construed as limiting. Various modifications and changes may be made to the subject matter described herein without following the example embodiments and applications illustrated and described, and without departing from the true spirit and scope of the present invention, which is set forth in the following claims.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

Aster R H. Blood platelet kinetics and platelet transfusion. J Clin Invest. 2013; 123(11):4564-5. doi: 10.1172/JC170335. PubMed PMID: 24177466; PMCID: PMC3809797.
Andrews R K, Berndt M C. Bernard-Soulier syndrome: an update. Semin Thromb Hemost. 2013; 39(6):656-62.
Becker G A, Tuccelli M, Kunicki T, Chalos M K, Aster R H. Studies of platelet concentrates stored at 22 C nad 4 C. Transfusion. 1973; 13(2):61-8. PubMed PMID: 4695593.
Bergmeier W, Bouvard D, Eble J A, Mokhtari-Nejad R, Schulte V, Zirngibl H, Brakebusch C, Fassler R, Nieswandt B. Rhodocytin (aggretin) activates platelets lacking alpha(2)beta(1) integrin, glycoprotein VI, and the ligand-binding domain of glycoprotein Ibalpha. J Biol Chem. 2001; 276(27):25121-6. doi: 10.1074/jbc.M103892200. PubMed PMID: 11352922.
Berndt M C, Gregory C, Chong B H, Zola H, Castaldi P A. Additional glycoprotein defects in Bernard-Soulier's syndrome: confirmation of genetic basis by parental analysis. Blood. 1983; 62(4):800-7. PubMed PMID: 6309285.
Brass L F, Shattil S J, Kunicki T J, Bennett J S. Effect of calcium on the stability of the platelet membrane glycoprotein IIb-IIIa complex. J Biol Chem. 1985; 260(13):7875-81. PubMed PMID: 2409078.
Brecher M E, Hay S N, Rothenberg S J. Evaluation of a new generation of plastic culture bottles with an automated microbial detection system for nine common contaminating organisms found in PLT components. Transfusion. 2004; 44(3):359-63. PubMed PMID: 14996192.
Brecher M E, Hay S N. Transfusion medicine illustrated. Platelet swirling. Transfusion. 2004; 44(5):627. doi: 10.1111/j. 1537-2995.2004.03428.x. PubMed PMID: 15104639.
Chen W, Druzak S A, Wang Y, Josephson C D, Hoffmeister K M, Ware J, Li R. Refrigeration-Induced Binding of von Willebrand Factor Facilitates Fast Clearance of Refrigerated Platelets. Arterioscler Thromb Vasc Biol. 2017; 37(12):2271-9. doi: 10.1161/ATVBAHA. 117.310062. PubMed PMID: 29097365; PMCID: PMC5699930.
Chen W, Liang X, Syed A K, Jessup P, Church W R, Ware J, Josephson C D, Li R. Inhibiting GP1balpha Shedding Preserves Post-Transfusion Recovery and Hemostatic Function of Platelets After Prolonged Storage. Arterioscler Thromb Vasc Biol. 2016; 36(9): 1821-8. doi: 10.1161/ATVBAHA.116.307639. PubMed PMID: 27417583; PMCID: PMC5001887.
Clemetson K J, McGregor J L, James E, Dechavanne M, Luscher E F. Characterization of the platelet membrane glycoprotein abnormalities in Bernard-Soulier syndrome and comparison with normal by surface-labeling techniques and high-resolution two-dimensional gel electrophoresis. J Clin Invest. 1982; 70(2):304-11. PubMed PMID: 6284798; PMCID: PMC371237.
Devine D V, Serrano K. The platelet storage lesion. Clin Lab Med. 2010; 30(2):475-87.
Egidi M G, D'Alessandro A, Mandarello G, Zolla L. Troubleshooting in platelet storage temperature and new perspectives through proteomics. Blood Transfus. 2010; 8 Suppl 3:s73-81. doi: 10.2450/2010.012S. PubMed PMID: 20606754; PMCID: PMC2897204.
Hoffmeister K M, Felbinger T W, Falet H, Denis C V, Bergmeier W, Mayadas T N, von Andrian U H, Wagner D D, Stossel T P, Hartwig J H. The clearance mechanism of chilled blood platelets. Cell. 2003; 112(1):87-97. PubMed PMID: 12526796.
Hoffmeister K M, Josefsson E C, Isaac N A, Clausen H, Hartwig J H, Stossel T P. Glycosylation restores survival of chilled blood platelets. Science. 2003; 301(5639): 1531-4.
Jackson C W, Krance R A, Edwards C C, Whidden M A, Gauthier P A. Shortened platelet survival as a cause of thrombocytopenia in mice with L1210 leukemia. Cancer Res. 1980; 40(3):667-70. PubMed PMID: 7471085.
Kanaji T, Russell S, Ware J. Amelioration of the macrothrombocytopenia associated with the murine Bernard-Soulier syndrome. Blood. 2002; 100(6):2102-7. doi: 10.1182/blood-2002-03-0997. PubMed PMID: 12200373.
Kattlove H E, Alexander B. The effect of cold on platelets. I. Cold-induced platelet aggregation. Blood. 1971; 38(1):39-48. PubMed PMID: 4254206.
Kuehnert M J, Roth V R, Haley N R, Gregory K R, Elder K V, Schreiber G B, Arduino M J, Holt S C, Carson L A, Banerjee S N, Jarvis W R. Transfusion-transmitted bacterial infection in the United States, 1998 through 2000. Transfusion. 2001; 41(12):1493-9. PubMed PMID: 11778062.
Kumar P, Satchidanandam V. Ethyleneglycol-bis-(beta-aminoethylether)tetraacetate as a blood anticoagulant: preservation of antigen-presenting cell function and antigen-specific proliferative response of peripheral blood mononuclear cells from stored blood. Clin Diagn Lab Immunol. 2000; 7(4):578-83. PubMed PMID: 10882655; PMCID: PMC95917.
Manning K L, Novinger S, Sullivan P S, McDonald T P. Successful determination of platelet lifespan in C3H mice by in vivo biotinylation. Lab Anim Sci. 1996; 46(5):545-8. PubMed PMID: 8905588.
Mason K D, Carpinelli M R, Fletcher J I, Collinge J E, Hilton A A, Ellis S, Kelly P N, Ekert P G, Metcalf D, Roberts A W, Huang D C, Kile B T. Programmed anuclear cell death delimits platelet life span. Cell. 2007; 128(6):1173-86. doi: 10.1016/j.cell.2007.01.037. PubMed PMID: 17382885.
McGill M, Brindley D C. Effects of storage on platelet reactivity to arterial subendothelium during blood flow. J Lab Clin Med. 1979; 94(2):370-80. PubMed PMID: 458253.
Murphy S, Gardner F H. Effect of storage temperature on maintenance of platelet viability—deleterious effect of refrigerated storage. N Engl J Med. 1969; 280(20):1094-8. doi: 10.1056/NEJM196905152802004. PubMed PMID: 5778424.
Nurden A T, Caen J P. Specific roles for platelet surface glycoproteins in platelet function. Nature. 1975; 255(5511):720-2. PubMed PMID: 1169691.
Odell T T, Jr., Mc D T. Life span of mouse blood platelets. Proc Soc Exp Biol Med. 1961; 106:107-8. PubMed PMID: 13730268.
Palavecino E L, Yomtovian R A, Jacobs M R. Bacterial contamination of platelets. Transfus Apher Sci. 2010; 42(1):71-82. doi: 10.1016/j.transci.2009.10.009. PubMed PMID: 19939734.
Rock G, Figueredo A. Metabolic changes during platelet storage. Transfusion. 1976; 16(6):571-9. PubMed PMID: 11576.
Rumjantseva V, Grewal P K, Wandall H H, Josefsson E C, Sorensen A L, Larson G, Marth J D, Hartwig J H, Hoffmeister K M. Dual roles for hepatic lectin receptors in the clearance of chilled platelets. Nat Med. 2009; 15(11):1273-80. doi: 10.1038/nm.2030. PubMed PMID: 19783995; PMCID: PMC4428152.
Savoia A, Kunishima S, De Rocco D, Zieger B, Rand M L, Pujol-Moix N, et al. Spectrum of the mutations in Bernard-Soulier syndrome. Hum Mutat. 2014; 35(9): 1033-45.
Sandrock-Lang K, Wentzell R, Santoso S, Zieger B. Inherited platelet disorders. Hamostaseologie. 2016; 36(3): 178-86.
Shively J A, Gott C L, De Jongh D S. The effect of storage on adhesion and aggregation of platelets. Vox Sang. 1970; 18(3):204-15. PubMed PMID: 5445621.
Slichter S J, Harker L A. Preparation and storage of platelet concentrates. II. Storage variables influencing platelet viability and function. Br J Haematol. 1976; 34(3):403-19. PubMed PMID: 10956.
Standards for blood banks and transfusion services. QRB Qual Rev Bull. 1977; 3(12):17,22. PubMed PMID: 414187.
Stubbs J R, Tran S A, Emery R L, Hammel S A, Haugen A L, Zielinski M D, Zietlow S P, Jenkins D. Cold platelets for trauma-associated bleeding: regulatory approval, accreditation approval, and practice implementation-just the “tip of the iceberg”. Transfusion. 2017; 57(12):2836-44. doi: 10.1111/trf.14303. PubMed PMID: 28880362.
Valeri C R. Circulation and hemostatic effectiveness of platelets stored at 4 C or 22 C: studies in aspirin-treated normal volunteers. Transfusion. 1976; 16(1):20-3. PubMed PMID: 1251454.
van der Meer P F. PAS or plasma for storage of platelets? A concise review. Transfus Med. 2016; 26(5):339-42. doi: 10.1111/tme.12325. PubMed PMID: 27273164.
Wandall H H, Hoffmeister K M, Sorensen A L, Rumjantseva V, Clausen H, Hartwig J H, Slichter S J. Galactosylation does not prevent the rapid clearance of long-term, 4 degrees C.-stored platelets. Blood. 2008; 111(6):3249-56. doi: 10.1182/blood-2007-06-097295. PubMed PMID: 18096766.
Ware J, Russell S, Ruggeri Z M. Generation and rescue of a murine model of platelet dysfunction: the Bernard-Soulier syndrome. Proc Natl Acad Sci USA. 2000; 97(6):2803-8. doi: 10.1073/pnas.050582097. PubMed PMID: 10706630; PMCID: PMC16010.
White J G. Effects of ethylenediamine tetracetic acid (EDTA) on platelet structure. Scand J Haematol. 1968; 5(4):241-54. PubMed PMID: 4969536.
Winokur R, Hartwig J H. Mechanism of shape change in chilled human platelets. Blood. 1995; 85(7):1796-804. PubMed PMID: 7703486.
Xiang B, Zhang G, Guo L, Li X A, Morris A J, Daugherty A, Whiteheart S W, Smyth S S, Li Z. Platelets protect from septic shock by inhibiting macrophage-dependent inflammation via the cyclooxygenase 1 signalling pathway. Nat Commun. 2013; 4:2657. doi: 10.1038/ncomms3657. PubMed PMID: 24150174; PMCID: PMC4217311.
Xiang B, Zhang G, Liu J, Morris A J, Smyth S S, Gartner T K, Li Z. A G(i)-independent mechanism mediating Akt phosphorylation in platelets. J Thromb Haemost. 2010; 8(9):2032-41. doi: 10.1111/j. 1538-7836.2010.03969.x. PubMed PMID: 20586915; PMCID: PMC2965800.
Xiang B, Zhang G, Stefanini L, Bergmeier W, Gartner T K, Whiteheart S W, Li Z. The Src family kinases and protein kinase C synergize to mediate Gq-dependent platelet activation. J Biol Chem. 2012; 287(49):41277-87. doi: 10.1074/jbc.M112.393124. PubMed PMID: 23066026; PMCID: PMC3510826.
Xiang B, Zhang G, Ye S, Zhang R, Huang C, Liu J, Tao M, Ruan C, Smyth S S, Whiteheart S W, Li Z. Characterization of a Novel Integrin Binding Protein, VPS33B, Which Is Important for Platelet Activation and In Vivo Thrombosis and Hemostasis. Circulation. 2015; 132(24):2334-44. doi: 10.1161/CIRCULATIONAHA.115.018361. PubMed PMID: 26399659; PMCID: PMC4679702.
Zhang G, Xiang B, Dong A, Skoda R C, Daugherty A, Smyth S S, Du X, Li Z. Biphasic roles for soluble guanylyl cyclase (sGC) in platelet activation. Blood. 2011; 118(13):3670-9. doi: 10.1182/blood-2011-03-341107. PubMed PMID: 21803853; PMCID: PMC3186338.
Zhang R, Zhang G, Xiang B, Chen X, Tang L, Shi S, Liu Y, Ai X, Xie P, Li Z. TRAF3 negatively regulates platelet activation and thrombosis. Sci Rep. 2017; 7(1):17112. doi: 10.1038/s41598-017-17189-1. PubMed PMID: 29215030; PMCID: PMC5719392.

1. An in vitro platelet composition comprising platelets and one or more Ca⁺⁺ chelators.

2. The platelet composition of claim 1, wherein the platelet composition comprises platelet rich plasma (PRP).

3. The platelet composition of claim 2, wherein the PRP is prepared from mammalian blood.

4. The platelet composition of claim 1, wherein the one or more Ca⁺⁺ chelators are ethylenediaminetetraacetic acid (EDTA), ethylene glycol-bis(p-aminoethyl ether)-N,N,N′,N′-tetraacetic acid (EGTA), fructose 1, 6-bisphosphate (FBP), 1,2-bis(o-aminophenoxy)ethane-N,N,N′,N′-tetraacetic acid (BAPTA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), citric acid, or citrate ions.

5. A method of storing the platelet composition of claim 1, wherein the method comprises storing the composition at a temperature above about 0° C. and less than about 22° C.

6. A method of maintaining hemostatic function of a platelet composition, wherein the method comprises adding one or more Ca⁺⁺ chelators to a platelet composition and storing the platelet composition at a temperature above 0° C. and less than about 22° C.

7. The method of claim 6, wherein the platelet composition comprises platelet rich plasma (PRP).

8. The method of claim 7, wherein the PRP is prepared from mammalian blood.

9. The method of claim 6, wherein the one or more Ca⁺⁺ chelators are ethylenediaminetetraacetic acid (EDTA), ethylene glycol-bis(p-aminoethyl ether)-N,N,N′,N′-tetraacetic acid (EGTA), fructose 1, 6-bisphosphate (FBP), or 1,2-bis(o-aminophenoxy)ethane-N,N,N′,N′-tetraacetic acid (BAPTA).

10. The method of claim 6, wherein the platelet composition is stored in the cold for five to 15 days.

11. The method of claim 6, wherein the platelet composition is stored at 4° C.

12. A method of treating a subject in need of platelet transfusion, wherein the method comprises transfusing the subject with the platelet composition of claim 1.

13. The method of claim 12, wherein the subject has low platelet count, abnormal platelet morphology, or abnormal platelet function.

14. The method of claim 12, wherein the subject has thrombocytopenia or a sport injury.

15. A method of inhibiting bleeding in a subject, wherein the method comprises administering to the subject the composition of claim 1.

16. The method of claim 15, wherein the blood loss is reduced by about 25%, 30%, 45%, 50%, 55%, 60%, 65%, 70%, 80% or 85% as compared to blood loss using RT-stored platelet without added Ca⁺⁺ chelators.

17. A method for monitoring transfused platelets in circulation, wherein the method comprises obtaining platelets from a transgenic animal that expresses a marker, injecting the platelets into a recipient animal, and monitoring and/or detecting the transfused platelets in circulation.

18. The method of claim 17, wherein the recipient animal is of the same species as the transgenic animal.

19. The method of claim 17, wherein the marker is GFP.

20. The method of claim 19, wherein the transgenic animal is a GFP mouse and the recipient animal is a mouse.
